(19) United States
(12) Patent Application Publication
(10) Pub. No.: US 2009/0062877 A1
(43) Pub. Date: Mar. 5, 2009

(54) Multisite heart pacing with adjustable number of pacing sites for terminating high frequency cardiac arrhythmias

(76) Inventors: Valentin Krinski, Villeneuve Loubet (FR); Stefan Luther, Gottingen (DE); Eberhard Bodenschatz, Gottingen (DE)

Correspondence Address: VALENTIN KRINSKI, 33 AVE DES SOURC , APT 5, VILLENEUVE LOUBEL, F-06270, FR

(21) Appl. No.: 12/040,007

(22) Filed: Feb. 29, 2008

Related U.S. Application Data
(60) Provisional application No. 60/892,855, filed on Mar. 3, 2007.

(51) Int. Cl. A61N 1/365

(52) U.S. Cl. 607/15

(57) ABSTRACT

A multisite heart pacing with adjustable number of pacing sites is realized by using only one lead directly connected to the heart. The number and locations of pacing sites is regulated by increasing the amplitude of pacing pulses delivered by the electric field, and by changing orientation of the electric field.

Improved termination of high frequency cardiac arrhythmias and AF is achieved by
regulating the number of pacing sites by choosing the pulse energy in the range 1/400-½ DE, where DE is energy of conventional cardioversion/defibrillation.
protection against inducing VF by choosing the direction and amplitude of the electric field, and by a proper synchronization with R wave of the ECG.
selection of the pacing frequency and amplitude based on the frequency spectrum of a high frequency cardiac arrhythmia.

### REFERENCES CITED

### US Patent References

U.S. Pat. No. 7,127,292 October 2006 Warman et al. Addressing recurrent atrial fibrillation (AF pacing)
U.S. Pat. No. 7,120,490 October 2006 Chen et al. Atrial shock timing optimization
U.S. Pat. No. 7,020,517 March 2006 Welner et al. Fibrillation/tachycardia preventive system
U.S. Pat. No. 7,006,867 February 2006 Kroll Overdrive pacing from multiple atrial sites
U.S. Pat. No. 7,142,928 November 2006 Sharma et al. Stimulation near a blade cut in the myocardium
U.S. Pat. No. 5,489,293 February 1996 Pless et al. Method and apparatus for treating cardiac tachycardia
U.S. Pat. No. 5,275,621 January 1994 Mehra Method and apparatus for Terminating tachycardia
U.S. Pat. No. 5,275,621 January 1994 Mehra Method and apparatus for Terminating tachycardia

### Foreign Patent References

| | | |
|---|---|---|
| 0 393 265 | October, 1990 | EP |
| 1 062 971 | December, 2000 | EP |
| 2 025 236 | January, 1980 | GB |

### Other Publications

Sepulveda, N G, Roth, B J, Wikswo, J P. Current injection into a two-dimensional anisotropic bidomain. Biophys J, 55(5), 987-99, 1989.
Allessie M, et al. Regional control of atrial fibrillation by rapid pacing in conscious dogs. Circulation. 1991; 84:1689-1697.
Daoud E G et al. Response of Type I Atrial Fibrillation to Atrial Pacing in Humans. Circulation. 1996; 94:1036-1040.
Disertori M, et al. Antitachycardia pacing therapies to terminate atrial tachyarrhythmias: the AT500 Italian Registry. European Heart Journal Supplements. 2001; 3:16-24.
A. Pumir, V. Krinsky, Unpinning of a rotating wave in cardiac muscle by an electric field. J. Theor. Biol, 199, 311-319, 1999.
S. Takagi, et al. Unpinning and removal of a rotating wave in cardiac muscle Phys. Rev. Let. 2004, 93 (5), 058101.

### DESCRIPTION

The numbers in claims and descriptions below are given not in a restrictive sense, but to illustrate the preferred embodiment of the invention. E.g., the same approach can be realized not with only one lead connected to the heart as we claim here, but more leads can give the same results as well.

### FIELD OF THE INVENTION

The present invention relates generally to a method for termination high frequency cardiac arrhythmias and, in particular, to a method for termination paroxysmal atrial fibrillation (AF) by multi site pacing where the number of pacing sites is regulated by changing amplitude and orientation of the electric field.

### BACKGROUND OF THE INVENTION

The only successful method to terminate high frequency cardiac arrhythmias is cardioversion/defibrillation. It has several important drawbacks. A discharge of a defibrillator in a conscious patient is painful and extremely unpleasant. It has also potential damaging effects.

On the other hand, exists a much more gentle method—antitachycardia pacing (ATP). It is not painful, its energy is several orders of magnitude less than that of cardioversion/defibrillation. ATP is successful against low frequency arrhythmias only (frequency not larger than 4 Hz). Its success rate decreases fast with increasing frequency of the arrhythmia, and high frequency cardiac arrhythmias (frequencies larger 4 Hz) and atrial fibrillation (AF) cannot be terminated by ATP.

Allessie (1991) has tried to entrain AF. He found it is possible only locally, in a small vicinity (several cm) of the pacing electrode; outside of it AF is not entrained.

An evident solution is to pace AF from so many sites that they cover atria dense enough. But many implanted pacing leads and their connecting wires would severely damage a contracting heart.

A method for terminating high frequency arrhythmias and AF is needed that uses an energy level much lower than that of conventional cardioversion/defibrillation and can terminate arrhythmias that ATP cannot terminate. It is desirable the energy level be below the pain threshold.

### SUMMARY OF THE INVENTION

The present invention satisfies a need for a technique that permits to terminate high frequency arrhythmias and, in particular, AF with a pulse energy much smaller than that of cardioversion/defibrillation.

We suggest to use heterogeneities naturally existing in the heart as pacing sites. Pacing from heterogeneities naturally existing in the heart has advantages over conventional pacing
1. a multisite pacing can be achieved without connecting many electrodes to the heart.
2. the number and position of pacing sites can be regulated by changing the amplitude and the direction of the electric field.
3. energy of the electric field pulse needed for this is 2-400 times smaller than that used in cardioversion/defibrillation.

We propose a method and apparatus for terminating high frequency arrhythmias—anti fibrillation pacing (AFP). An implanted device for AFP and an external device for AFP are disclosed.

In heart preparations, AFP terminates high frequency cardiac arrhythmias and AF with pulses of much smaller energy than the cardioversion/defibrillating pulse, and with much higher success rate than conventional ATP.

This summary of the invention and the advantages and features thereof have been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

FIG. 1: The larger size R′ of an obstacle, the larger is depolarization e′ₘₐₓ induced near it by an electric field Dimensionless coordinates: Obstacle size R′=R/λ, where R is obstacle size in mm, λ˜0.5 mm is the electronic constant of the tissue. Depolarization e′ₘₐₓ=e/eₘₐₓ, where e is depolarization in mV, eₘₐₓ is depolarization near a very large obstacle. An analytical solution of the linearized model (Pumir, Krinsky, 1999).

FIG. 2: Increasing the number of pacing sites by increasing the electric field (a-c). Modifying positions of pacing sites by changing direction of the electric field (d,e).

(a-c)—electric field is increased. a) E=0.45 V/cm—pacing from 1 electrode. Short arrows indicate direction of the wave propagation. The pacing wave looks like a moon since it is initiated near a circular obstacle. Far from it, the pacing wave has a circular shape, as usual. b) E=0.47 V/cm—pacing from 2 electrodes. c) E=0.58 V/cm—pacing from 4 electrodes.

(d,e)—direction of the electric field (long arrow) is changed. Amplitude of the electric field is the same E=0.5 V/cm. Numerical simulation of the LR model.

FIG. 3: Pacing from heterogeneities (virtual electrodes) removes rotating waves. t=0.04 s: R1 and R2—rotating waves. t=0.18 s: P—a pacing wave emitted from a heterogeneity (a white circle) by a pulse of Electric field E=1.25 V/cm. t=0.92 s: the tip R2 approaches the front of pacing wave and collides with it.

t=0.96 s: the tip R2 disappeared but a new wave break is formed on the front of R1. Thus, a jump of the wave break position and its orientation (arrows) was induced (compare arrows at t=0.92 s and t=0.96 s). t=1.24 s: Rotating wave R2 is terminated. R1 is in the position to be terminated by the same mechanism. t=1.38 s: Rotating waves are removed. Pacing waves emitted from the heterogeneity (a white circle at the upper right corner) entrain the whole medium. Numerical simulation of the LR model.

FIG. 4: Pacing from an ATP fixed lead does not remove rotating waves. t=0.04 s: same as in FIG. 3. t=0.28 s: P—pacing wave 1 emitted from a fixed electrode (right lower corner). Dashed line is a boundary of the region paced by this wave. t=0.42 s: pacing wave 2: the size of the paced region is increased. t=0.54 s: pacing wave 3 decays. A fuzzy front is seen instead of a sharp front observed at propagating waves. t=1.32 s: size of the paced region is decreased (compared to t=0.42 s). t=2.36 s: Rotating waves are not removed. A small paced region (at the lower right corner) enlarges and shrinks quasi periodically. Numerical simulation of the LR model. All parameters are same as in FIG. 3

FIG. 5: A diagram illustrating an embodiment of controlling high frequency cardiac arrhythmias by AFP external device. 3—defibrillating electrode, 4—diagnostic catheter, 6—catheter with stimulating electrodes, 8—paddle electrodes, 11—ECG electrodes, 17—switch.

FIG. 6: A flow chart illustrating AFP external device. 3—defibrillating electrode, 4—diagnostic catheter, 13, 14, 15—controls to set manually the period of the stimulating pulses, the number of pulses and the energy of each pulse, 17,18—switches.

FIG. 7: A flow chart illustrating AFP implanted device. 3—defibrillating electrode, 18—switch, 25—stimulating electrode, 26—defibrillating electrode, 27—sensing electrode.

### PHYSICAL PRINCIPLES USED IN THE INVENTION

The present invention satisfies the need for a technique that permits to use pacing for termination high frequency arrhythmias, in particular, AF.

Numerous natural heterogeneities existing in the heart are used here as pacing sites. The size distribution of natural heterogeneities in the heart is wide: from microns to millimeters. This permits to control number of pacing sites from 1-2 to dozens.

The physical mechanism is well known in cardiology: it is a change of membrane potential by an electric field near defects. This phenomenon was given name “Virtual electrodes” (Sepulveda at al, 1989).

Virtual electrodes are believed to play an important role in defibrillation, exciting all tissue and thus terminating all propagating waves. Creating a large size virtual electrode by cutting the cardiac tissue with a blade was proposed in U.S. Pat. No. 7,142,928 in order to decrease almost twice the pacing threshold from a small fixed wire electrode.

An electric field, applied to the heart creates depolarized and hyperpolarized regions near every heterogeneity, corresponding to redistributions of the intracellular and extracellular currents. If the induced depolarization is above the threshold, it can induce a propagating excitation wave. This mechanism was used in cardiology to explain how defibrillation works. We propose to use this effect for creating as many pacing sites as needed, from 1-2 to dozens. We verified in experiments with cardiac muscle preparations that 1-2 pacing sites were induced by a pulse of an electric field as low as 0.15-0.25 V/cm, 3-5 pacing sites—with 0.25-0.35 V/cm, dozens pacing sites—with 0.35-0.5 V/cm. To compare, electric field needed for defibrillation is huge, ˜6 V/cm. The electric field E needed to induce 3-5 pacing sites is ˜20 times less than that of defibrillation, needed to induce dozens pacing sites is more than 10 times less than that of defibrillation. Thus, in our proposed method, the pulse energy W(W˜E²) may be hundreds times less than that of defibrillation.

A more detailed description of the mechanism making our proposed method more powerful than ATP and requiring much less energy than defibrillation is below.

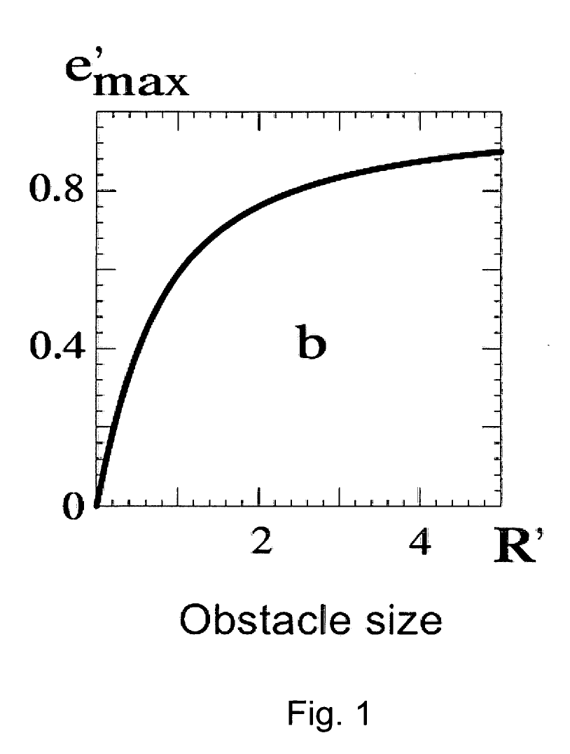

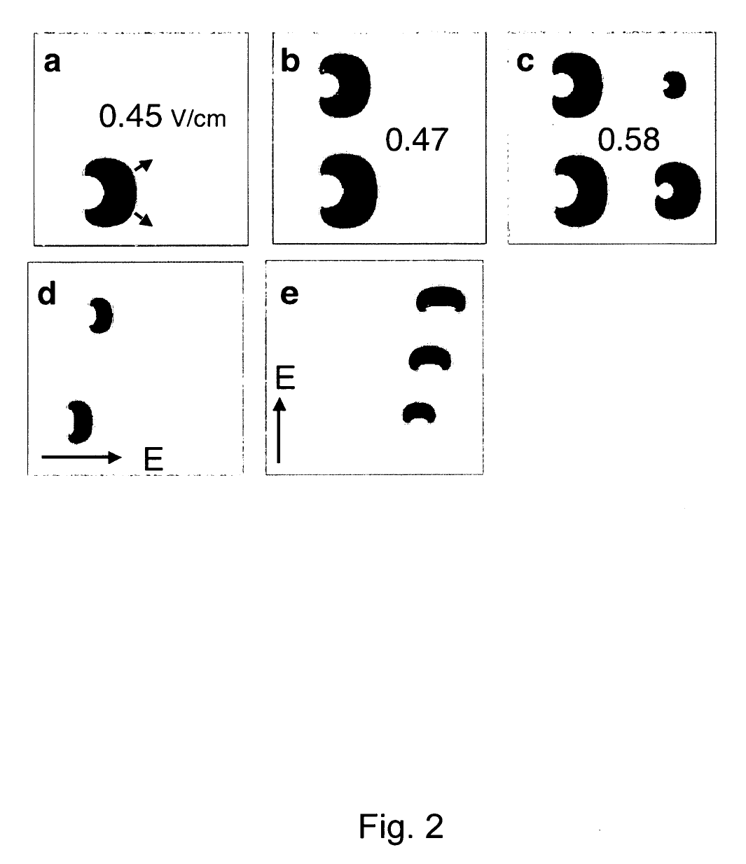

The larger size of the obstacle, the larger is depolarization induced by an electric field near it (FIG. 1). Pulses of electric field of small amplitude induce pacing only from the large size heterogeneities (FIG. 2 a). Increasing amplitude of the electric field induces pacing from smaller and smaller size heterogeneities (FIG. 2 b, c). For obstacles of generic shape (not circular), orientation of the electric field affects the position and the number of pacing sites (FIG. 2 d,e). FIGS. 1 and 2 demonstrate that

(i) increasing the intensity of the applied electric field with a fixed direction leads to wave emission from an increasingly large set of heterogeneities in the tissue.

(ii) changing the direction of the applied electric field leads to wave emission from different sets of heterogeneities in the tissue.

This permits to realize a multisite heart pacing with adjustable number of pacing sites. Increasing the number and changing location of pacing sites can be achieved by increasing the amplitude of pacing pulses delivered by the electric field, and by changing orientation of the electric field.

A multisite heart pacing with adjustable number of pacing sites that we propose, results in an improved method for termination high frequency cardiac arrhythmias and AF. We call this method ‘anti fibrillation pacing’ (AFP). Let us compare our method with ATP.

The conventional ATP is successful only against low frequency arrhythmias, and its success rate decreases fast with increasing frequency of the arrhythmia. The physical mechanism behind the ATP inability to terminate high frequency arrhythmias is:
with low frequency pacing, all pacing waves propagate over the whole heart.
but with high frequency pacing, the propagation of the high frequency waves cannot be sustained.

The high frequency waves decay with distance. Due to the Wenckebach rhythm transformation, generically every second wave decays (more rare, every third wave decays). Thus, only near the pacing electrode, the frequency of the propagating waves is the frequency of pacing; at a distance, the frequency of propagating waves becomes lower. The low frequency waves can capture only the low frequency arrhythmias, but not the high frequency arrhythmias.

To terminate a high frequency pathological source of waves, the pacing electrode should be situated close to it. With conventional fixed pacing leads, this can be achieved by chance only. Pacing from cardiac heterogeneities permits to regulate the number and the position of pacing sites, and thus to avoid this problem.

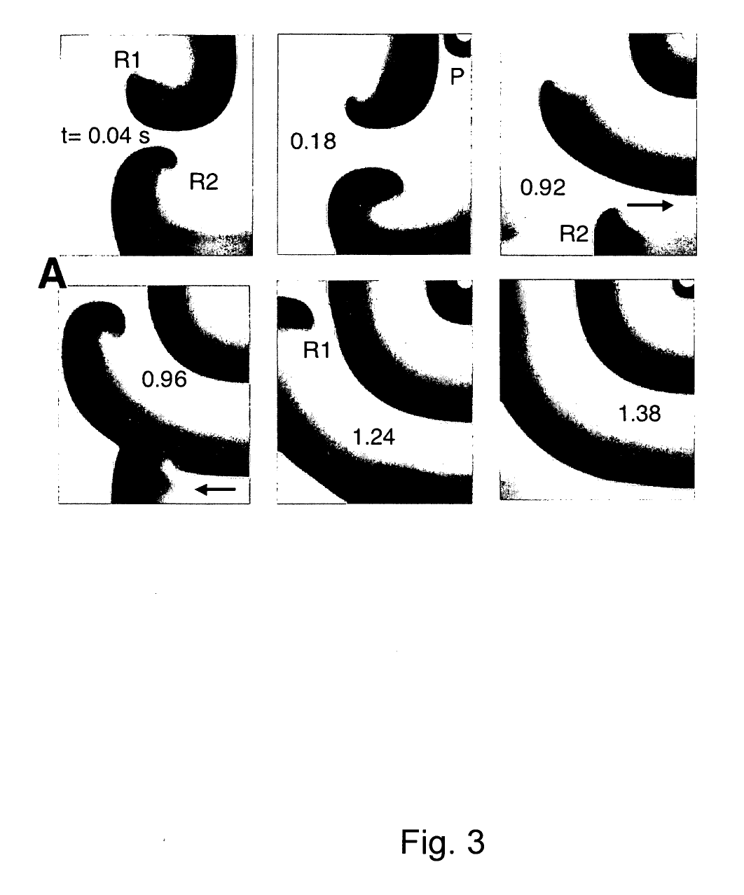

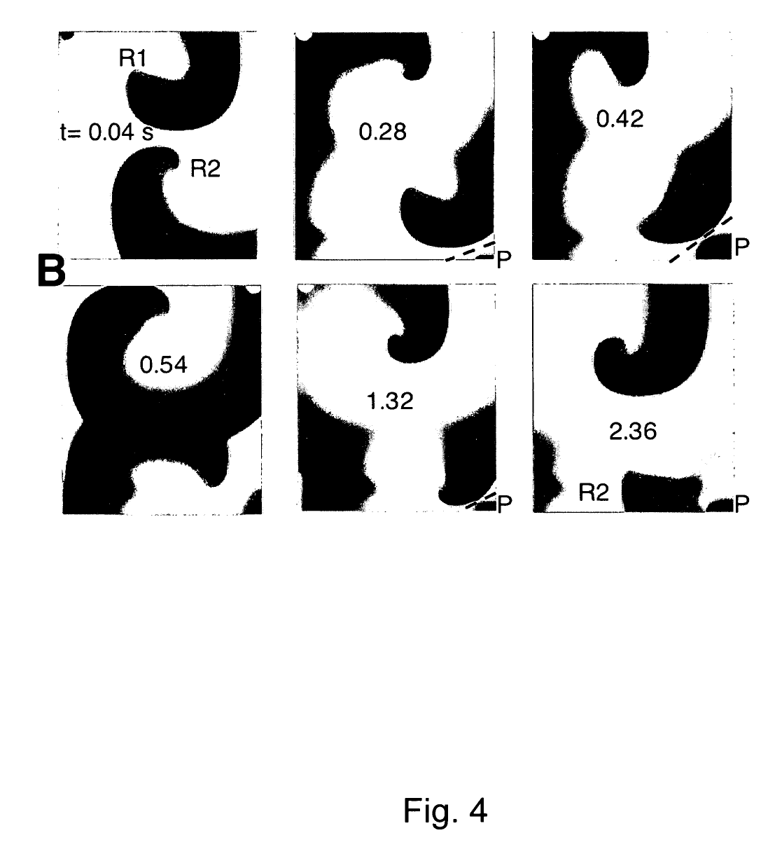

We illustrate numerically in FIGS. 3, 4 how, for geometrical reasons, a conventional fixed pacing lead may fail to pace away a set of rotating waves, whereas virtual electrodes in the tissue permits to pace away a set of rotating waves. On FIG. 3, only one heterogeneity used for pacing is shown.

### The AFP Device

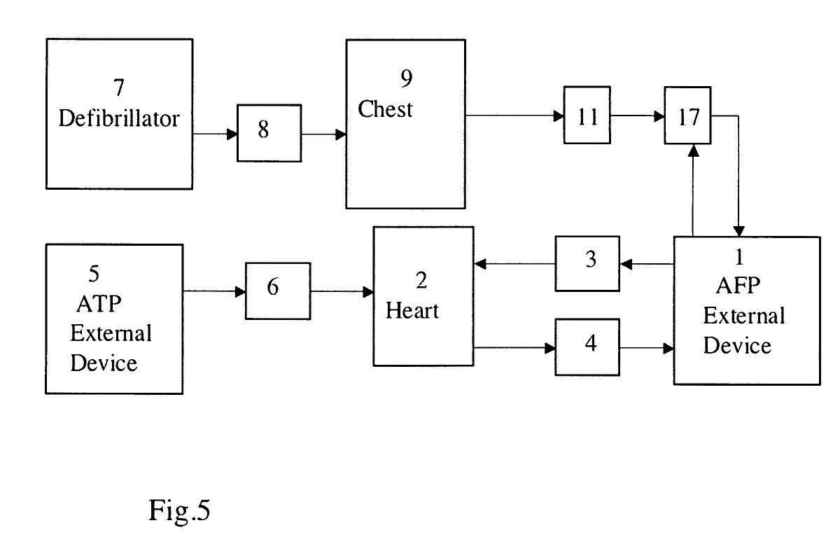

An embodiment for AFP external device is shown in FIG. 5. The device for controlling high frequency cardiac arrhythmias consists of the following main parts: Pulse generating block 12. Arrhythmia Frequency spectrum analyzer 16, Pulse Energy selector 21, Pacing Frequency selector 22.

Pulse generating block 12 is tuned by controls 13, 14, 15 to set manually the period of the pulses, the number of pulses and the energy of a pulse. Pulse generating block 12 is connected to defibrillating electrodes 3, to switch 18 and to memory 20. The Pulse generating block 12 is different from the cardioverter/defibrillator and the ATP pacemaker: it is able to deliver pulses with time interval much shorter than usually needed to charge the defibrillators capacitor, and to pace from defibrillating electrodes, it should supply 1-2 orders of magnitude less pulse energy than a defibrillator.

Defibrillating electrode: In a preferred embodiment of the invention, it is an intracardiac defibrillating electrode, a catheter. In second preferred embodiment, it may be an implanted intracardiac electrode. External defibrillating patches can be used as well, but not for the case where pulses below the pain threshold are needed.

Pulse Energy selector 21 obtains data from ECG/EMG amplifier 10 and is connected to the memory 20. Pacing Frequency selector 22 obtains data from ECG/EMG amplifier 10 and is connected to the memory 20. All of them obtain data from Memory as well and send data to Monitor with recommendations to clinician the values to be set for the period of the pulses, the number of pulses and the energy of a pulse.

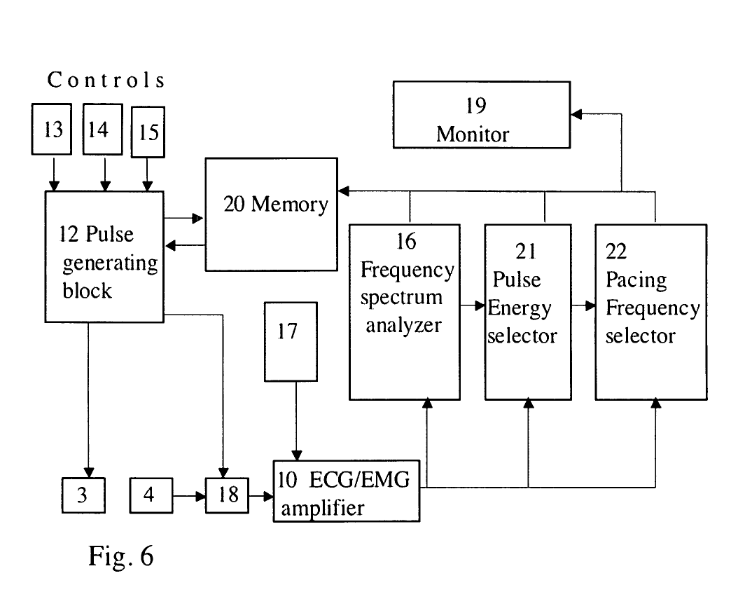

More details are shown in FIG. 6. Arrhythmia Frequency spectrum analyzer 16 obtains data from ECG/EMG amplifier 10 and is connected to the memory 20. Frequency Spectrum analyzer 16 is intended to

a) help to choose the pacing frequency for pacing from virtual electrodes

b) protect from delivering an electric field pulse near the T wave on the ECG.

Function a) is needed since during AF, the EMG/ECG records are not periodic, and choice of the pacing interval even by a well trained medical personnel may be erroneous. In a stationary device, the analyzer supplies on line frequencies and amplitudes of 3 largest peaks in the Fourier spectrum of the arrhythmia, and the whole Fourier spectrum.

Function b) is intended for an additional protection against induction of VF when AFP is applied in atria. Usually, synchronization of the cardioverter/defibrillator with the R wave is used. But since AFP delivers several electric field pulses, all of them cannot be synchronized with the R wave. Instead, the EMG/ECG automatic analyzer for AFP protects from delivering an electric field pulse near the T wave on the ECG.

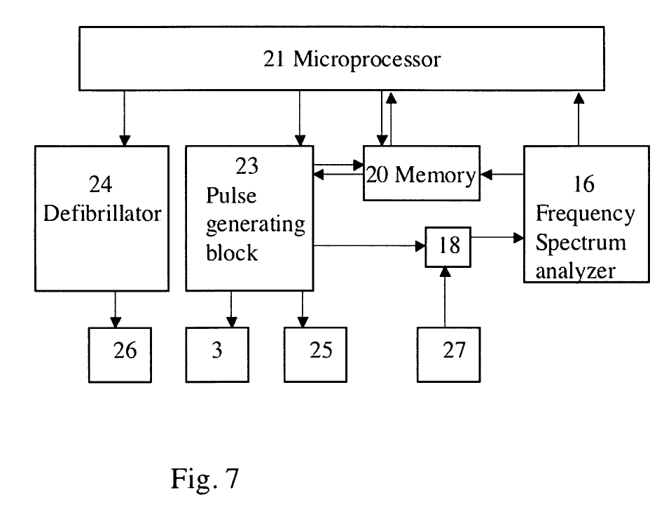

Flow chart of an implanted device is shown in FIG. 7. Pulse generating block 23 delivers pulses either from an implanted ATP electrode 25 or from defibrillation electrodes 3. For pacing from an implanted electrode, it chooses standard pacing amplitude applied for ATP.

For pacing from defibrillation electrodes, it permits to choose the energy of pulses from interval: 0.01 J-1 J for intracardiac defibrillating electrodes, the time interval between pulses 100 ms-250 ms, and the number of pulses 4-8 pulses.

Pulse generating block 23 receives data from microprocessor 21 and operates a switch 18. In an implanted device, Frequency Spectrum analyzer 16 contains several band pass filters to avoid overloading of a microprocessor with calculations of Furrier spectrum. Frequency Spectrum analyzer 16 obtains data from sensing electrode 27 and sends data to memory 20 and to the microprocessor 21. Microprocessor 21 selects Pulse Energy and Pacing Frequency and sends these data to the Pulse generating block 23. Microprocessor 21 operates also defibrillator 24.

### DETAILED DESCRIPTION OF THE PREFERRED EMBODIMENT OF THE INVENTION

The preferred embodiment of the invention is: an external AFP device, used for clinical investigations. An arrhythmia with frequency higher than that permitting to use ATP is detected. The arrhythmia frequency is above the threshold for ATP not more than by 50%.

FIG. 5 is a diagram illustrating an embodiment of controlling high frequency cardiac arrhythmias by anti-fibrillation pacing (AFP) external device. AFP External Device 1 is coupled to a patient's heart 2 with diagnostic catheter 4 and defibrillating electrode 3, that may be either an implanted defibrillating electrode or a catheter. ATP external device 5 is connected to a patient's heart 2 via catheter 6 with monopolar or bipolar stimulating electrodes. Defibrillator 7 is connected to the patient chest 9 via paddle electrodes 8. ECG amplifier 10 is connected via switch 17 to ECG electrodes 11. Switch 17 disconnects ECG amplifier 10 from ECG electrodes 11 when AFP external device 1 delivers AFP pulses to the heart.

FIG. 6 is a flow chart illustrating AFP external device. Pulse generating block 12 is connected to defibrillating electrode 3. Controls 13, 14, 15 are connected to Pulse generating block 12. They permit to set manually the period of the stimulating pulses (control 13), the number of pulses (control 14) and the energy of each pulse (control 15). Frequency spectrum analyzer 16 is connected to ECG amplifier 10 and diagnostic catheter via switch 18. Switch 18 disconnects ECG/EMG amplifier 10 from diagnostic catheter 4 when Pulse generating block 12 delivers AFP pulses to the heart.

Frequency spectrum analyzer 16 is connected to monitor 19 where it displays the Fourier spectrum of AF or other high frequency arrhythmia to help medical personnel to chose pacing frequency for ATP or AFP.Pulse generating block 12 and Frequency spectrum analyzer 16 are connected to memory 20.

AFP External Device 1 may be realized as a box containing all these elements, or it can use an external PC as ECG/EMG analyzer 16, memory 20 and monitor 19.

FIG. 7 is a flow chart illustrating AFP implanted device. Pulse generating block 23 is connected to the heart via defibrillating electrode 3 when it delivers AFP pacing and via stimulating electrode 25 when it delivers ATP pacing. Defibrillator 24 is connected to heart via defibrillating electrode 26 which may be same as defibrillating electrode 3. Frequency spectrum analyzer 16 is connected to heart via sensing electrode 27. All is controlled by Microprocessor 21.

This summary of the invention and the advantages and features thereof have been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### CONCLUSION

While particular embodiments of the invention have been disclosed herein in detail, this has been done for the purposes of illustration only, and is not intended to limit the scope of the invention as defined in the claims that follow. It is to be understood that various substitutions, alterations, or modifications can be made to the disclosed embodiment without departing from the spirit and scope of the claims. The above described implementations are simply those presently preferred or contemplated by the inventors, and are not to be taken as limiting the present invention to the disclosed embodiments. It is therefore to be understood, that within the scope of the appended claims, the invention may be practiced otherwise than as specifically described without actually departing from the spirit and scope of the present invention.

1. A method and apparatus for cardiac multi site pacing with adjustable number of pacing sites using only one lead directly connected to the heart. Increasing the number and changing location of pacing sites is achieved by increasing the amplitude of pacing pulses delivered by the Electric field, and by changing orientation of the Electric field.

2. A method and apparatus for termination high frequency cardiac arrhythmias and fibrillation (short name: antifibrillation pacing, AFP), comprising a block (e.g. microchip) for automatic determination of the frequency spectrum of a high frequency cardiac arrhythmia consisting e.g. of 3-7 band pass filters, a block (e.g. microchip) selecting the pacing pulse energy in the range 1/400-½ of the standard defibrillation energy (DE), a block (e.g. microchip) selecting the AFP pacing frequency (e.g. in the range 0.9-1.1 the arrhythmia dominant frequency), and pulse generating block delivering 4-8 AFP pulses from intracardiac defibrillation electrodes or patches.

3. The method and apparatus of claim 2 further comprising a protection against induction of ventricular fibrillation (VF): positioning of both defibrillating electrodes to minimize the current flow into ventricles (for atrial pacing); and the energy threshold less than the low level of vulnerability (LLV).
